(19) United States
(12) Patent Application Publication
(10) Pub. No.: US 2026/0007927 A1
(43) Pub. Date: Jan. 8, 2026

(54) METHOD FOR GUIDED AND MONITORED EXERCISE ROUTINES

(71) Applicant: The Hong Kong Polytechnic University, Hung Hom (HK)

(72) Inventors: Pui Ching Shirley NGAI, Hung Hom (HK); Man Hin LAM, Hung Hom (HK); Wui Man LAU, Hung Hom (HK); Ching Hang FONG, Hung Hom (HK); Ho Keung David SHUM, Hung Hom (HK)

(73) Assignee: The Hong Kong Polytechnic University, Hung Hom (HK)

(21) Appl. No.: 19/007,014

(22) Filed: Dec. 31, 2024

Related U.S. Application Data
(60) Provisional application No. 63/667,003, filed on Jul. 2, 2024.

(51) Int. Cl. A63B 24/00; A61B 5/00; A61B 5/08; A63B 71/06

(52) U.S. Cl. CPC A63B 24/0075; A61B 5/08; A61B 5/7246; A63B 71/0622; A63B 2024/0068

(57) ABSTRACT

A method for guided and monitored exercise routines wherein physiological data measurements are used to determine an optimized exercise program for a participant. The exercises are displayed on a smart device in such a way as to maximize the benefits given the participant's current physiological state. Improvements including wellness monitoring and safety monitoring steps are also disclosed. Measurements of total and individual lung performance are used to select exercises and monitor health.

### CROSS-REFERENCE TO RELATED APPLICATIONS

This application claims priority from and the benefit of U.S. Provisional Patent Application 63/667,003 filed Jul. 2, 2024 in the United States Patent & Trademark Office. All disclosures of the document(s) named above are incorporated herein by reference.

### BACKGROUND OF THE INVENTION

### 1. Field of the Invention

Aspects of the invention relate to exercise routines for a participant guided by an external device, the external device having access to telemetry of the participant's physiological state such as heart rate, respiratory rate, and other parameters as shall be shown herein.

### 2. Description of the Related Art

Various “smart” devices such as smart watches, smart mirrors, and smart exercise equipment can measure either directly or indirectly the physiological conditions of the participant while exercising. However, the existing format of exercise prescription via an online or app platform often has limited ability to prescribe or make exercise progression according to the participant's physiological responses (including but not limited to heart rate or respiratory rate).

This type of exercise prescription has substantial limitations in tailoring the exercise type and level of difficulty (or exercise intensity) according to the condition of the participants, which reduces its benefits. A method for evaluating the condition of the participants and adjusting the exercise type and level of difficulty or intensity would be a useful invention.

Such exercise prescription can also pose safety risks to the participants as there is no monitoring of their performance and physiological responses. A method for evaluating the condition of the participants for health and safety issues during exercise would be a useful invention.

As respiratory infections and conditions which can affect lung health and efficiency are now a common and serious health and safety issue, a method for monitoring lung health and efficiency over time would also be a useful invention.

Aspects of the present invention address these concerns.

### SUMMARY OF THE INVENTION

Aspects of the present invention comprise a method for using a collection of medical sensors and a smart device which can receive data from these sensors to both evaluate the physiological condition of a participant using the method and design, to implement, and to guide the participant through exercises suitable for their condition while monitoring the participant for any irregularities or health and safety issues during the exercises. In particular, volumetric evaluation of lung efficiency and consistency can be monitored as part of the execution of the method.

Additional aspects and/or advantages of the invention will be set forth in part in the description which follows and, in part, will be obvious from the description, or may be learned by practice of the invention.

### BRIEF DESCRIPTION OF THE DRAWINGS

These and/or other aspects and advantages of the invention will become apparent and more readily appreciated from the following description of the embodiments, taken in conjunction with the accompanying drawings of which:

FIG. 1 is a front view of a device used in the execution of the method of the invention.

FIG. 2 is a front view of a smartphone being used in the execution of the method of the invention.

FIG. 3 is a flow chart of the method of the invention.

FIG. 4 is an abstract schematic of the device used in the execution of the method of the invention.

### DETAILED DESCRIPTION OF THE EMBODIMENTS

Reference will now be made in detail to several embodiments of the invention illustrated in the accompanying drawings. Whenever possible, the same or similar reference numerals will be used in the drawings and descriptions to refer to the same or like parts or steps. The drawings are in simplified form and are not to precise scale. For purposes of convenience and clarity only, directional terms such as top, bottom, left, right, up, down, over, above, below, beneath, rear, and front, will be used with respect to the drawings. These and similar directional terms are not to be construed to limit the scope of the invention in any manner. The words attach, connect, couple, and similar terms with their inflectional morphemes do not necessarily denote direct or intermediate connections, but can also include connections through mediate elements or devices.

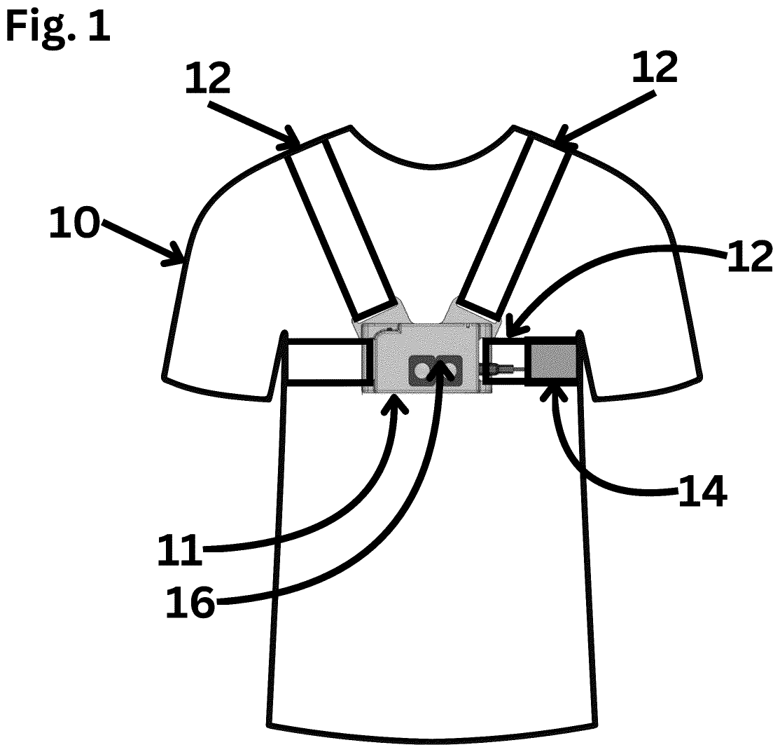

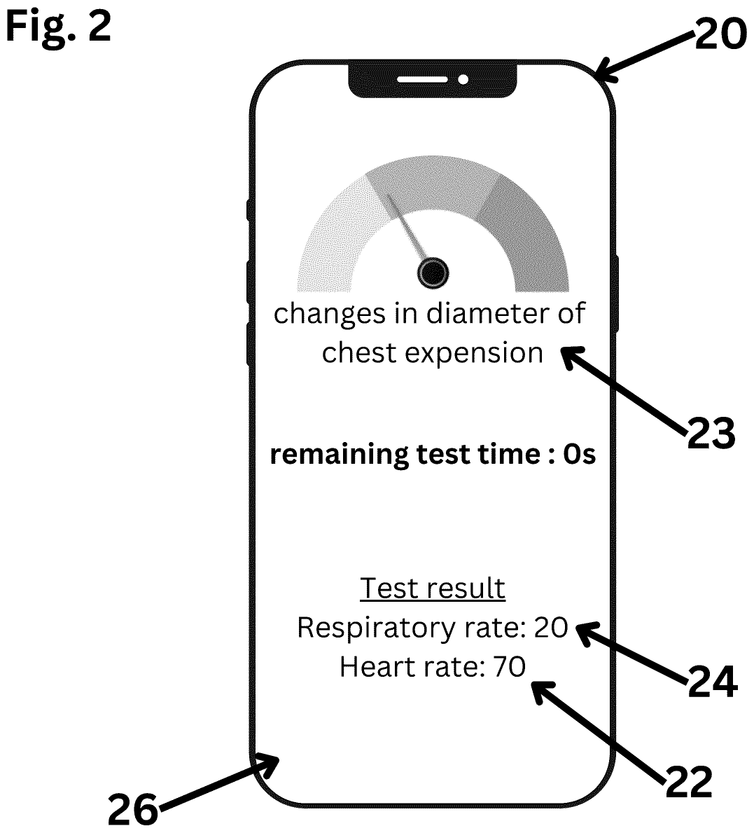

By referring to FIGS. 1 & 2, the basic principle of the invention can be easily understood. FIG. 1 shows the torso of a participant wearing a sensor rig used in the execution of the method. Participant 10 is wearing sensor rig 11, which includes straps 12, chest expansion sensors 14, and control unit 16. Control unit 16 can incorporate and/or be connected to additional sensors (NOT SHOWN) including but not limited to: a heart rate monitoring device, and/or any other appropriate or desired sensors. Control unit 16 includes a wireless send/receive device such as a Bluetooth® transceiver, a proprietary wireless transceiver, and/or any other appropriate or desired send/receive device. Physiological state data is gathered by sensor rig 11 and transmitted to smart device 20 (see FIG. 2). Straps 12 are optional, though some reasonable means for attaching sensor rig 11 to the participant is useful. The sensors can be integrated directly into control unit 16 or connected to it by wire or wirelessly. A power source (not shown) such as a disposable or rechargeable battery is also required, but any appropriate source(s) would be familiar to those of ordinary skill in the art and so are not further described or specified.

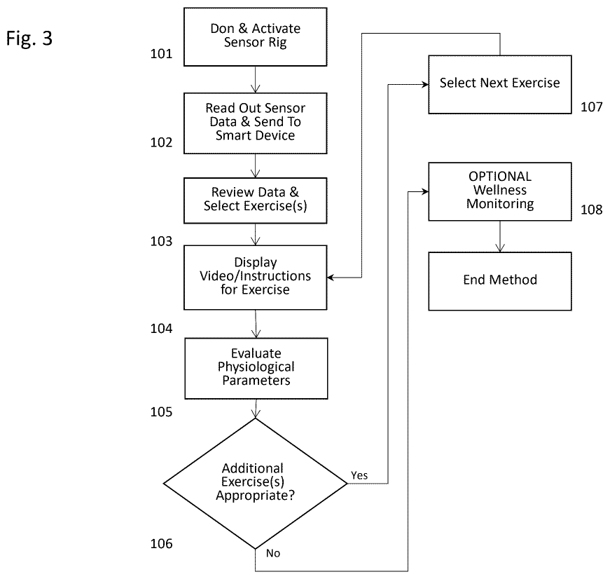

As shown in FIG. 2, smart device 20 receives the data from sensor rig 11 and uses it to execute the method of the invention. (See FIG. 3.) Smart device 20 may comprise a cell phone, a tablet computer, a laptop, a desktop computer, a smart watch, or any other appropriate or desired device. In the embodiment described herein, sensor rig 11 uses data from chest expansion sensors 14 to display the respiratory rate of the participant on display 26, e.g. as respiratory rate readout 24, but by using multiple sensors, can display the readout of heart rate 22 and the diameter changes of chest expansion readout 23.

It is also possible to have one or more sensors built into the smart device, such as an accelerometer, a gyroscope, a step sensor, etc., during the execution of the method of the invention. While the description of the embodiment will assume that all sensors are incorporated into the sensor rig, the method described will work equally well using such smart device sensors during its execution.

FIG. 3 shows the steps of the execution of the method of the invention. In Step 101, the participant dons and activates the sensor rig 11, including powering sensor rig 11 on, powering on the smart device 20 if needed, linking sensor rig 11 to the smart device 20 if needed, and observing the smart device 20 to wait for an indication that the smart device 20 is receiving data from the sensor rig 11.

In Step 102, the sensor rig 11 reads out the current parameters for all sensors present and activated. If motion or other activity is necessary on the part of the participant, the smart device displays instructions and/or examples. For instance, if the common “sit to stand” exercise is used to measure one or more parameters, the smart device 20 will indicate to the participant to sit down and stand up one or more times, including an optional display of a video of a person performing the desired action (s.)

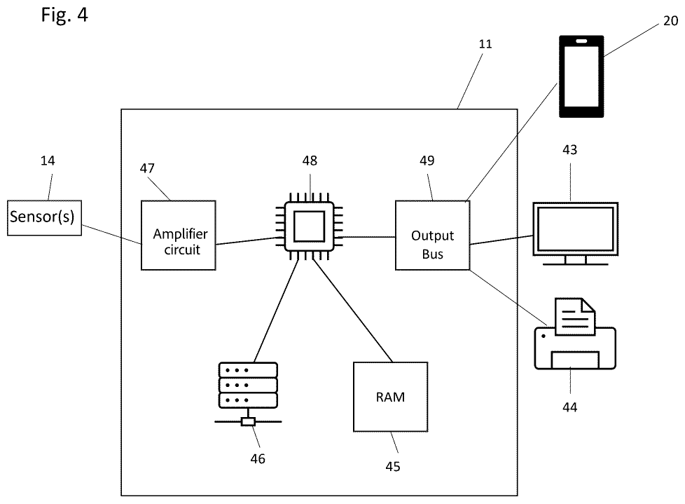

In Step 103, the smart device 20 executes a program (see FIG. 4) which collates the data gathered in Step 102 and correlates it against other known parameters of the participant such as their age, past medical history, etc., to determine an appropriate set of one or more exercises tailored for maximum efficiency in improving the participant's health while remaining safely within their capabilities. The program can be configured to emphasize one or more parameters as appropriate. As an example, if the participant's heart rate is suboptimal, the program can select an exercise or exercises meant to improve pulmonary function and capacity. The program integrates the assessment results, physiological data and subjective exertion collected for the prescription of exercise, including a combination of warm-up, breathing exercises, strengthening exercises, aerobic exercises, and stretching exercises as appropriate. The first exercise selected becomes a current exercise.

In Step 104, the current exercise is displayed on the smart device 20 using text, audio, and/or video prompting to allow the participant to most effectively perform the exercise for maximum efficiency and benefits.

In Step 105, after each exercise component, respiratory rate, heart rate, and other physiological parameters of the participant are measured to decide the progression of the exercise routine using a custom algorithm.

In Step 106, if additional exercises are determined to be appropriate by the program, a next exercise is selected in Step 107 as determined by the custom algorithm and which becomes the current exercise, otherwise the method ends.

In Step 107, the method returns to Step 104 with the current exercise, and the method repeats from Steps 104 to 106.

In optional wellness monitoring step 108, some or all of the data gathered during the execution of the method of the invention is directly transmitted and/or stored for later transmission or review to health professionals. For instance, if the participant's respiratory rate has increased after exercising as determined by the individual chest expansion sensors, a physician can be notified, and further diagnostic procedures are initiated.

An optional safety monitoring step 109 (NOT SHOWN) can also be executed during execution of the method. If any physiological parameter measured by sensor rig 11 leaves a defined safe range, including but not limited to unsafe heart rate, lung expansion, or body temperature, one or more additional steps are executed. This step of the method is performed as frequently as desired, either between other measurements and evaluations or concurrently with the same data. If an unsafe condition is detected, the smart device 20 pauses or halts the exercise program until the unsafe condition ceases. It is optional to have the sensor rig 11 continue to monitor the physiological data and instruct the smart device 20 to resume the method once all parameters return to a safe range.

FIG. 4 shows an abstract schematic for a device capable of executing the method of an aspect of the invention. Sensor rig 11 comprises a central processing unit or CPU 48, a persistent storage 46, a random access memory or RAM 45, an amplifier circuit 47, and an output bus 49. Sensor(s) such as sensor 14 are connected to sensor rig 11 and then amplified by the amplifier circuit 47. Amplifier circuit 47 sends the sensor data to CPU 48, which processes it appropriately and stores it in RAM 45 and/or persistent storage 46 as directed by control code stored in persistent storage 46. Persistent storage 46 can comprise a solid state drive or SSD, a hard disk drive or HDD, flash storage, or any other reasonable and appropriate storage means.

Smart device 20, here a smartphone, has a communications connection to output bus 49. This can be wired or wireless as set forth above. The program which embodies the method of the invention can be stored as an app on smart device 20 and/or in the persistent storage and/or RAM of sensor rig 11.

Any portion of the program which embodies the method of the invention stored on smart device 20 is executed by it on the data received from sensor rig 11. It is optional to also send the data to a printer 44, a separate display 43, or a cloud-based transmission system (NOT SHOWN) as would be apparent to a person of ordinary skill in the art. The data, the evaluation of the data, and the exercises to be performed are then displayed on the display of smart device 20 and/or separate display 43. The data, the evaluation, and/or instructions for the exercise(s) can also be printed on printer 44.

Although a few embodiments of the present invention have been shown and described, it would be appreciated by those skilled in the art that changes may be made in this embodiment without departing from the principles and spirit of the invention, the scope of which is defined in the claims and their equivalents.

What is claimed is:

1. A method for guided and monitored exercise routines comprising:
a) operably affixing at least one sensor element to a participant;
b) activating the sensor element to collect at least one base physiological data measurement from the participant;
c) evaluating the base physiological data measurement to select at least one targeted exercise for the participant;
d) selecting a current targeted exercise from the set of at least one targeted exercise;
e) displaying the current targeted exercise on a display such that the participant can follow along and perform the current targeted exercise;
f) collecting at least one new physiological data measurement after completion of the current targeted exercise;
g) determining whether at least one additional exercise is appropriate based on the evaluation of the new physiological data measurement and if so proceeding to the next step, otherwise ending the method;
h) selecting a new targeted exercise from the one or more targeted exercises and making the new targeted exercise the current targeted exercise; and
i) returning to the step wherein the current targeted exercise is displayed such that the participant can follow along and perform the current targeted exercise.

2. The method for guided and monitored exercise routines of claim 1, further comprising:
j) recording the baseline physiological data measurement and/or the new physiological data measurement to create at least one recorded physiological data measurement.

3. The method for guided and monitored exercise routines of claim 2, wherein at least one recorded physiological data measurement is transmitted to a healthcare professional for evaluation.

4. The method for guided and monitored exercise routines of claim 1, further comprising:
j) determining at least one safety limit correlated with at least one monitored physiological data measurement, such that each monitored physiological data measurement has at least one correlated safety limit;
k) monitoring each physiological data measurement correlated to the correlated safety limit at predetermined intervals with at least one sensor; and
l) determining if any monitored physiological data measurement exceeds the correlated safety limit and, if so, halting the method.

5. The method for guided and monitored exercise routines of claim 4, further comprising:
m) determining whether the monitored physiological data measurement exceeds the correlated safety limit by a predetermined danger level and if so, sending an alert to one or more third party recipients.

6. The method for guided and monitored exercise routines of claim 4, further comprising:
m) recording the baseline physiological data measurement and/or the new physiological data measurement to create at least one recorded physiological data measurement for wellness monitoring purposes.

7. The method for guided and monitored exercise routines of claim 5, wherein the recorded physiological data measurement is transmitted to a healthcare professional for evaluation.

8. The method for guided and monitored exercise routines of claim 4, further comprising:
m) resuming the method after all monitored physiological data measurements return within the correlated safety limits.

9. The method for guided and monitored exercise routines of claim 5, further comprising:
n) resuming the method after all monitored physiological data measurements return within the correlated safety limits.

10. The method for guided and monitored exercise routines of claim 6, further comprising:
n) resuming the method after all monitored physiological data measurements return within the correlated safety limits.

11. The method for guided and monitored exercise routines of claim 7, further comprising:
n) resuming the method after all monitored physiological data measurements return within the correlated safety limits.

12. The method for guided and monitored exercise routines of claim 1, wherein at least one of the base physiological data measurements and/or at least one of the new physiological data measurements is a measurement of lung performance.

13. The method for guided and monitored exercise routines of claim 12, wherein the measurement of lung performance measures the performance of a right lung and a left lung as separate pulmonary data measurements.

14. The method for guided and monitored exercise routines of claim 2, wherein at least one of the physiological data measurements is a measurement of lung performance.

15. The method for guided and monitored exercise routines of claim 14, wherein the measurement of lung performance measures a right lung and a left lung as separate pulmonary data measurements.

16. The method for guided and monitored exercise routines of claim 3, wherein at least one of the physiological data measurements is a measurement of lung performance.

17. The method for guided and monitored exercise routines of claim 16, wherein the measurement of lung performance measures a right lung and a left lung as separate pulmonary data measurements.

18. The method for guided and monitored exercise routines of claim 4, wherein at least one of the physiological data measurements is a measurement of lung performance.

19. The method for guided and monitored exercise routines of claim 18, wherein the measurement of lung performance measures a right lung and a left lung as separate pulmonary data measurements.

20. The method for guided and monitored exercise routines of claim 5, wherein at least one of the physiological data measurements is a measurement of lung performance.

21. The method for guided and monitored exercise routines of claim 20, wherein the measurement of lung performance measures a right lung and a left lung as separate pulmonary data measurements.
